(19) United States
(12) Patent Application Publication
(10) Pub. No.: US 2021/0084971 A1
(43) Pub. Date: Mar. 25, 2021

(54) MULTI-CHANNEL VAPORIZER

(71) Applicant: Resurgent Pharmaceuticals, Inc., Minneapolis, MN (US)

(72) Inventor: Eric Greenbaum, Denver, CO (US)

(21) Appl. No.: 17/114,097

(22) Filed: Dec. 7, 2020

Related U.S. Application Data
(63) Continuation of application No. PCT/US2019/035767, filed on Jun. 6, 2019.
(60) Provisional application No. 62/681,265, filed on Jun. 6, 2018.

(51) Int. Cl. A24F 40/30; A24F 40/485; A24F 40/20; A24F 40/10; A24F 40/60; A24F 40/42; A61M 15/06; A61M 15/00

(52) U.S. Cl. CPC A24F 40/30; A24F 40/485; A24F 40/20; A24F 40/10; A24F 40/60; A61M 2205/8206; A61M 15/06; A61M 15/0021; A61M 15/0086; A61M 2205/502; A24F 40/42

(57) ABSTRACT

A multichannel vaporizer including multiple vapor sources and a user interface to modify a proportion of vapors from the vapor sources into a customized vapor to be delivered to the mouthpiece. Also, a method of providing a customized combination of vapors including adjusting a user interface to control an adjustable vapor manifold with a plurality of vapor paths defined between the plurality of vapor sources and a mouthpiece for issuing vapors.

### FIELD OF THE INVENTION

The invention relates generally to vaporizer devices for vaporizable products.

### BACKGROUND OF THE INVENTION

As more and more states legalize cannabis use for medical and recreational purposes new administration modalities have risen in popularity. One such administration modality is vaporization providing the inhalable substance substantially in the form of a vapor (i.e., a substance that is in the gas phase at a temperature lower than its critical point). These devices typically comprise a power source, such as a battery, actuated by a button-press, coupled to a vapor source of some kind. Vapor sources vary and include vaporizer cartridges containing a vaporizable cannabis extract, as well as heating elements or chambers used to vaporizer botanical material (dried flower) and cannabis extracts. The rise in popularity of this route of administration may be attributed to, inter alia, ease of use, variety of formulations, and discretion.

Prefilled vaporizer cartridges containing a cannabis extract, and cannabis extracts generally, are widely available in markets with legalized cannabis programs. There is a significant amount of variety in the types of formulations available for vaporization. Different formulations contain varying amounts of the major cannabinoids (THC and CBD) as well as various flavor enhancers such as terpenes, terpinoids, flavonoids and other flavoring agents. One of the benefits of such variety is that different vaporizable formulations will provide different pharmacological effects—enabling users to select a formulation to meet their needs at a given time.

One of the shortcomings in the vaporizer market is that in order to get different pharmacological effects, users must purchase several different vaporizable formulations. This can be both expensive and inconvenient. For example, if a medical cannabis patient wanted to have formulations containing 6 different THC to CBD ratios, that patient would have to purchase 6 different formulations, or undertake the difficult task of creating their own formulations. Therefore there is a need in the art for devices, systems, and methods to enable users to adjust the makeup of vaporizable formulations “on the fly” without having to change vaporizer cartridges or other otherwise change formulations.

### SUMMARY

As specified in the Background Section above, there is a need in the art to develop new vaporizer devices, formulations, and methods to provide a more titratable experience for users. Therefore, this disclosure provides descriptions of:
A multi-channel vaporizer having a plurality of vapor sources connected via a vapor manifold and vapor paths such that a user may adjust the ratio of vapor provided by each vapor source.
A vaporizer kit having a multi-channel vaporizer device, the device having: a power generation and regulation component; multiple vapor sources each containing a formulation adapted to be used with the vaporizer device; a vapor source engaging component; a vapor manifold; vapor pathways; a mouthpiece; and a vapor mixing controller; a charging device; and instructions for use.
An adjustable vapor manifold having vapor source engagement portions, at least one vapor path for communicating vapor from at least one vapor source to a mouthpiece, and at least one vapor regulator in communication with the at least one vapor path such that the vapor regulator can be adjusted to alter the ratio of vapor being delivered from each of the at least one vapor source thereby enabling a user to fine tune the composition of vapor.
A method of providing a user with titratable vapor dosing, the method having the steps of: providing a user with a vaporizer device having a plurality of vapor sources connected via a vapor manifold such that a user is capable of adjusting the ratio of vapor provided by each vapor source; and providing instruction on how to adjust the ratios of the vapors from the plurality of vapor sources.

A method for communicating the ratio of vapor composition to a user comprising the steps of providing a multichannel vaporizer with a plurality of vaporizable formulations in cartridges, wherein each formulation is associated with a dominant, distinctive, flavor profile so that the user associates that flavor profile with a given component of the vapor.

These and other needs and disadvantages are overcome by the methods, systems, and compositions of matter disclosed herein. Additional improvements and advantages may be recognized by those of ordinary skill in the art upon study of the present disclosure.

### BRIEF DESCRIPTION OF THE DRAWINGS

FIG. 1 illustrates by perspective view an exemplary implementation of a multichannel vaporizer in accordance with one embodiment of the present invention.

FIG. 2 illustrates by perspective view another embodiment of a vaporizer.

FIG. 3A illustrates by perspective view another embodiment of a vaporizer in accordance with the present invention.

FIG. 3B illustrates the vaporizer of FIG. 3A in a partially disassembled state.

FIG. 3C illustrates another view of the vaporizer of FIG. 3A in a partially disassembled state.

FIG. 4A illustrates by perspective view another embodiment of a hand-held multichannel vaporizer in accordance with the present invention.

FIG. 4B illustrates the vaporizer in FIG. 4A in a partially disassembled state.

FIG. 5 depicts components of an embodiment of a multichannel vaporizer in accordance with another embodiment of the present invention.

FIG. 6 depicts components of the vaporizer of FIG. 5.

FIG. 7 depicts components of another embodiment of a multichannel vaporizer in accordance with another embodiment of the present invention.

FIG. 8 depicts components of another embodiment of a multichannel vaporizer in accordance with another embodiment of the present invention.

FIG. 9 depicts components of another embodiment of a multichannel vaporizer in accordance with another embodiment of the present invention.

FIG. 10A illustrates in perspective view a multichannel vaporizer in accordance with the present invention.

FIG. 10B depicts a prior art vaporizer cartridge.

FIG. 11A, 11B, 11C illustrate in perspective views another embodiment of a multichannel vaporizer in accordance with the present invention.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context

The cannabinoids are a class of molecules primarily obtained through the extraction of cannabis plant material, although synthetic and/or bioreactor production may also be used. The various cannabinoids include tetrahydrocannabinol (THC), cannabidiol (CBD), (“the major cannabinoids”), tetrahydrocannabinolic acid (THCA), Cannabidiolic acid (CBDA), cannabinol (CBN), cannabigerol (CBG), cannabichromene (CBC), tetrahydrocannabivarin (THCV), and cannabidivarin (CBDV) as well as others (“the minor cannabinoids”). Various cannabinoids, used alone or in combination have shown a variety of significant biological effects including but not limited to pain relief, anticancer, anti-inflammatory, antiemetic, anticonvulsant, and many others, including recreational effects.

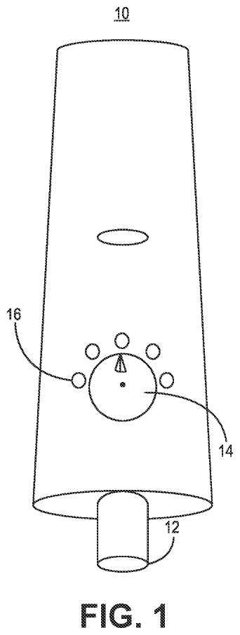

The Vaporizer: various embodiments of a multi-channel vaporizer are shown and described herein. For example, FIG. 1 shows an embodiment of a multi-channel vaporizer 10 having a mouthpiece 12, a manual vapor manifold controller 14 and ratio indicators 16 which provide information to the user on the ratio of vapor concentrations being provided. The inventors contemplated a variety of ratio indicators may be used. A color-coded labeling scheme that indicates specific ratios of cannabinoid content can rapidly provide information to users about the ratio of cannabinoids in the generated vapor. One color-coding scheme is described in applicants' U.S. patent application Ser. No. 15/436,487, herein incorporated in its entirety by reference. The manual manifold controller 14 depicted in FIG. 1 has discrete “stops” at the dots, which would be colored in an actual device, and which correspond to specified ratios of THC to CBD. For example: RED: 1:19; YELLOW: 6:1; GREEN: 1:1; BLUE: 1:6; and INDIGO: 1:19, THC to CBD.

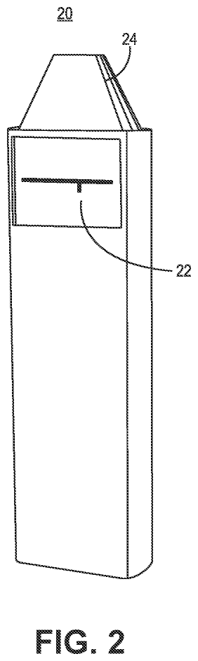

FIG. 2 shows another embodiment of a hand-held multi-channel vaporizer 20 with a different aesthetic style and illustrating the use of a touch screen mediated vapor manifold controller 22 shown in this example positioned near mouthpiece 24. In one example, vapor manifold controller 22 controls operation of an electronic valve(s) to control vapor flow out of two or more vapor sources, such as vapor cartridges. Internal vapor paths direct vapor from the valve(s) to mouthpiece 24. Depicted in this embodiment is a “slider” style user interface (UI) in which users slide a finger(s) along a touch screen thereby moving an indicator to a desired position along a color spectrum indicating the ratio of various cannabinoids. Touch screen UI may also provide additional control options such as, for example, overall potency, flavor profile, flavor strength, use control, use monitoring, lockouts, and generally providing an interface to computer enabled multi-channel vaporizer.

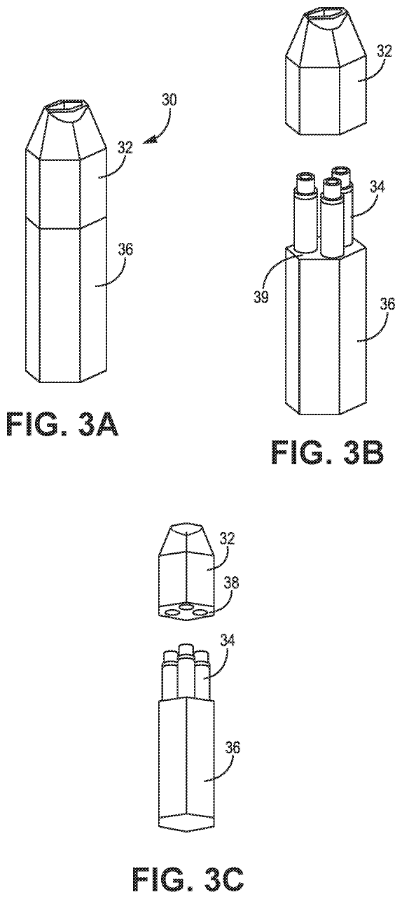

FIGS. 3A through 3C illustrate a multi-channel vaporizer 30 in accordance with one embodiment of the present invention. Vaporizer 30 is preferably a hand-held device including an upper mouthpiece portion 32 adapted to engage a plurality of vapor sources 34 and a bottom enclosure 36 comprising a power generation and control component, such as vaporizer batteries and/or “mods” known to those having skill in the art. In this example, vapor source 34 includes a plurality of separably-removable vapor cartridges. Bottom enclosure 36 further comprises a plurality of cartridge engaging portions.

FIGS. 3A through 3C depict a three cartridge vaporize model. Other examples include a two cartridge model and four or more cartridge models (not shown). The mouthpiece portion 32 further comprises vapor source engaging portions 38, an adjustable vapor manifold and a mouthpiece outlet. The bottom enclosure 36 further comprises a power generation and control component, such as vaporizer batteries and/or “mods” known to those having skill in the art. The bottom enclosure 36 further comprises a plurality of cartridge engaging portions 39. The mouthpiece portion 32 engages with the bottom enclosure 36 to assemble the multichannel vaporizer 30.

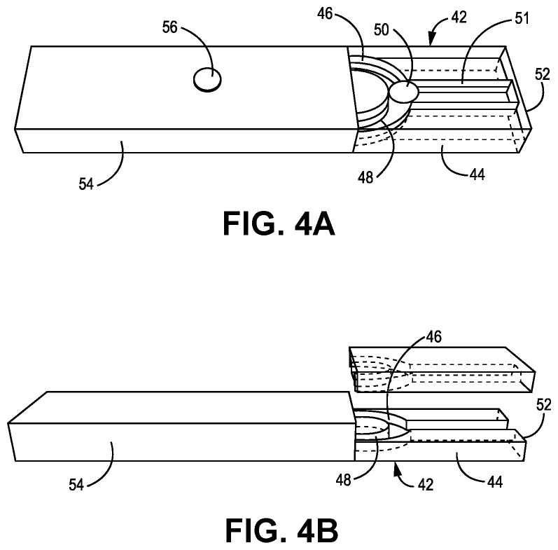

FIGS. 4A and 4B illustrates a two vapor source vaporizer 40 including an adjustable vapor control manifold 42 that is integrated with a top portion 44 of the multichannel vaporizer 40. In some embodiments the top portion 44 of the multichannel vaporizer 40 further comprise multiple molded vapor paths 46, 48 that converge into an adjustable mixing valve 50. An outlet vapor path 51 is provided between adjustable mixing valve 50 and an outlet of mouthpiece 52. Operation of mixing valve 50 is controlled via user interface 56. Vapor paths 46, 48 are each in fluid communication with a vapor source (not shown) housed within held-held enclosure 54. In this example, a pair of vapor sources are coupled to vapor paths 46, 48 leading to adjustable mixing valve 50. In some embodiments the top portion 44 is constructed of a transparent material such as glass or acrylic. Transparent top portions with integrated molded vapor paths have the advantage of users being able to see the vapor moving through the device. In some embodiments the multiple vapor paths are lined with an inert material such as ceramic, glass, quartz glass, titanium, surgical steel, or the like.

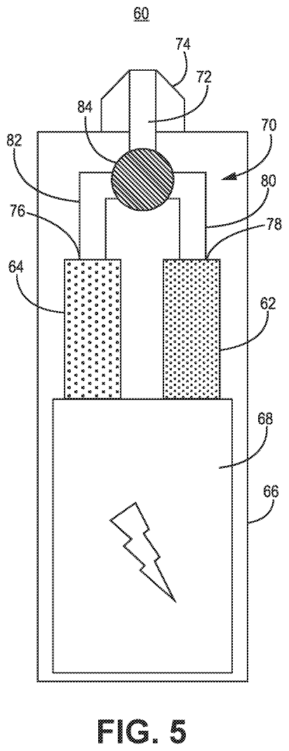

FIG. 5 depicts a cross sectional, schematic representation of an embodiment of a multichannel vaporizer 60 that contains two vapor source cartridges 62, 64. In general terms a hand-held housing 66 contains a power source, such as battery 68 and power source regulator, and a user interface. When assembled for use, the multichannel vaporizer 60 will also have at least one, but preferably at least two, vapor cartridges 62, 64 containing vaporizable formulations, engaged with cartridge engaging portions of the housing 66. Cartridges to be used in the multichannel vaporizer may include suitable vaporizer cartridges known to those having skill in the art. A controllable vapor manifold (“AVM”) 70 engages with the cartridges 62, 64 and also provides a vapor path 72 to a mouthpiece 74. The AVM 70 further comprises a plurality of cartridge engaging portions which, in some embodiments, engage the cartridges 62, 64 at their vapor output portions 76, 78. The AVM 70 further comprises vapor paths 80, 82 that create a vapor-tight path from the vapor cartridges 62, 64 to a vapor mixing portion 84. The vapor mixing portion 84 may be any vapor mixing means known to those having skill in the art, such as, for example a three-way mixing valve that may be manually- or computer-controlled. Vapor path 72 operatively connects to the outflow of the vapor mixing portion 84 and extends into mouthpiece 74 where the combined vapors may be inhaled by a user.

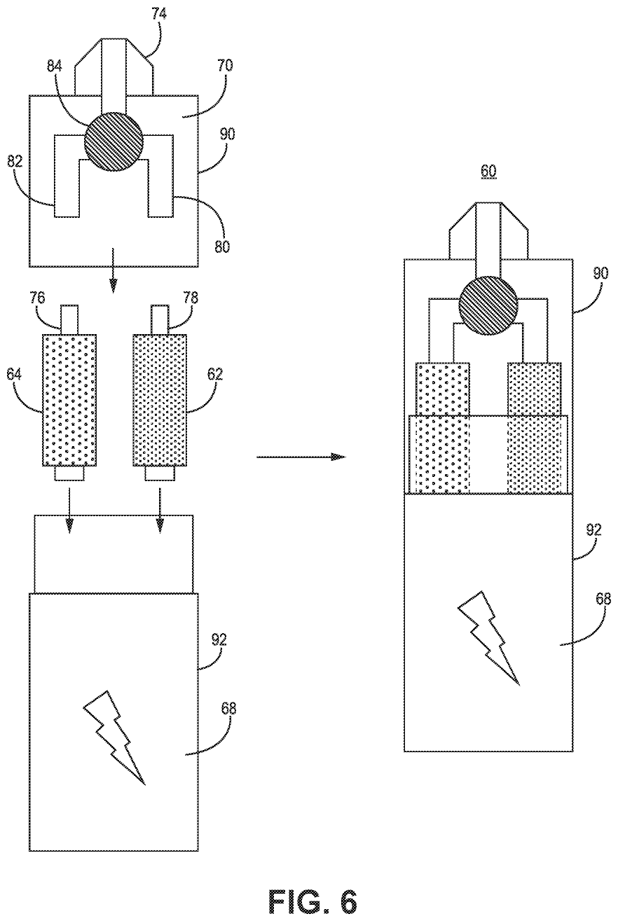

FIG. 6 illustrates a top/bottom configuration for an embodiment of a multichannel vaporizer 60. In this embodiment, the multichannel vaporizer 60 comprises a top portion 90 and a bottom portion 92 that come together to assemble the multichannel vaporizer 60. The ability to disengage the top portion 90 from the bottom portion 92 enables easy access to internal components such as vapor source cartridges 62, 64 and battery 68 housed as part of the bottom portion 92. When the top portion 90 of the multichannel vaporizer 60 is engaged with the bottom portion 92, cartridge engaging portions 94 of an AVM 70 engage the vapor output portions 96 of at least one but preferably a plurality of cartridges 62, 64. Top and bottom portions 90, 92 may engage each other via friction fitting, magnetic coupling, mechanical connector, or other engagement means known to those having skill in the art.

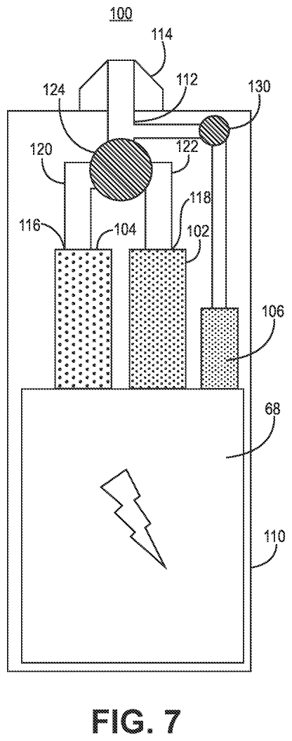

FIG. 7 shows a cross sectional, schematic representation of an embodiment of the multichannel vaporizer 100 that contains three vapor source cartridges 102, 104, 106. In general terms a bottom housing 110 contains a power generator and regulator, such as a battery 68, wherein the power regulator has cartridge engaging portions, such as “510” threaded sockets, magnet-engaging sockets, or the like. When assembled for use, the multichannel vaporizer 100 will also have at least one, but preferably, at least two, vapor source cartridges 102, 104 containing vaporizable formulations, engaged with the cartridge engaging portions. Cartridges, to be used in the multichannel vaporizer may any suitable vaporizer cartridge known to those having skill in the art and further comprising a vapor output portion. An adjustable vapor manifold (“AVM”) 70 engages with the cartridges 102, 104 and communicates with a vapor path 112 to a mouthpiece 114. The AVM 70 further comprises a plurality of cartridge engaging portions which, in some embodiments, engage the cartridges 102, 104 at vapor output portions 116, 118. The AVM 70 further comprises vapor paths 120, 122 that create a vapor-tight path from the cartridges 102, 104 to a vapor mixing portion 124. The vapor mixing portion 124 may be any vapor mixing means known to those having skill in the art, such as, for example a three-way mixing valve that may be manually or computer controlled. Vapor path 112 is operatively connected to the outflow of the vapor mixing portion 124 and extends into mouthpiece 114 where the vapor may be inhaled by a user.

FIG. 7 further illustrates a separate channel for cartridge 106. An embodiment such as this is suited for the delivery of a vapor with varying amounts of two primary formulations, held by the two larger cartridges 102, 104, and a tertiary formulation held in smaller cartridge 106. In specific embodiments the two primary formulations would be a THC formulation and CBD formulation while the tertiary formulation would be a blend of terpenes, flavonoids, and/or other ingredients designed to impart a pleasing or pharmacologically active flavor/taste profile. By having the tertiary formulation on a separate channel, i.e., controlled by a separate flow controller, the intensity of that vapor ingredient can be controlled independently from the primary formulations. For example, if a user wanted to change the flavor intensity of vapor, that user could manipulate (manually or electronically) the valve 130 leading from the mouthpiece vapor path 112 to the tertiary formulation cartridge 106. Alternatively, if a user wanted to maintain the flavor intensity of a vapor while altering the THC to CBD ratio that can be done by adjusting the mixing portion 124 between the primary vapor cartridges 102, 104 and the mouthpiece 114. In some embodiments, multiple tertiary formulations may be present in separate cartridges operatively connected to a mixing valve such that ratios of tertiary formulations can be adjusted while maintaining the same overall THC/CBD ratio/potency and flavor intensity.

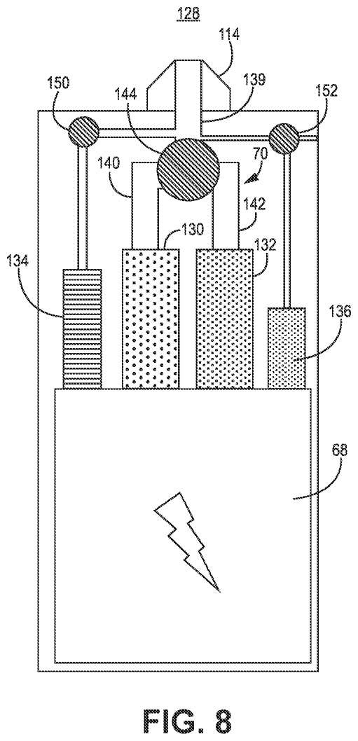

FIG. 8 shows a cross sectional, schematic representation of an embodiment of the multichannel vaporizer 128 that contains four separate vapor source cartridges 130, 132, 134, 136. In general terms a bottom housing 138 contains a power generator and regulator, such as a battery 68, wherein the power regulator has cartridge engaging portions, such as “510” threaded sockets, magnet-engaging sockets, or the like. When assembled for use, the multichannel vaporizer will also have at least one, but preferably, at least two, cartridges 130, 132 containing vaporizable formulations, engaged with the cartridge engaging portions. Primary cartridges 130, 132 to be used in the multichannel vaporizer 128 may any suitable vaporizer cartridge known to those having skill in the art and further comprising a vapor output portion. An adjustable vapor manifold (“AVM”) 70 engages with the cartridges 130, 132 and provides a vapor path 139 to a mouthpiece 114. The AVM 70 further comprises a plurality of cartridge engaging portions which, in some embodiments, engage the cartridges 130, 132 at their vapor output portions. The AVM 70 further comprises vapor paths 140, 142 that create a vapor-tight path from the cartridges to a vapor mixing portion 144. The vapor mixing portion 144 may be any vapor mixing means known to those having skill in the art, such as, for example a three-way mixing valve that may be manually or computer controlled. Vapor path 139 is operatively connected to the outflow of the vapor mixing portion 144 extends into mouthpiece 114 where the vapor may be inhaled by a user.

Further depicted in this example is separate channels for the 3^{rd} and 4^{th} cartridges, 134, 136. An embodiment such as this is suited for the delivery of a vapor with varying amounts of two primary formulations, held by the two larger cartridges 130, 132, a tertiary formulation and a quaternary formulation. In specific embodiments the two primary formulations would be a THC formulation and CBD formulation while the tertiary formulation would be a blend of terpenes, flavonoids, and/or other ingredients designed to impart a pleasing or pharmacologically active flavor/taste profile; and the quaternary formulation would be selected from the group of compounds referred to as the “minor cannabinoids.” By having the tertiary and quaternary formulations on separate channels, e.g., controlled by separate flow controllers, the intensity of that vapor ingredient can be controlled independently from the primary formulations. In some embodiments, multiple tertiary and/or quaternary formulations may be present in separate cartridges operatively connected to a mixing valve such that ratios of tertiary and quaternary formulations can be adjusted while maintaining the same overall THC/CBD ratio/potency and flavor intensity. Vapor flow through the tertiary and/or quaternary channels is controlled via valves 150, 152. Valve 152 may have a three-way mixing valve in order to draw in external air to balance vapor flows through the AVM 70. In this manner the amount of vapor from the tertiary and quaternary channels can be varied but the overall ratio between the channels could remain constant.

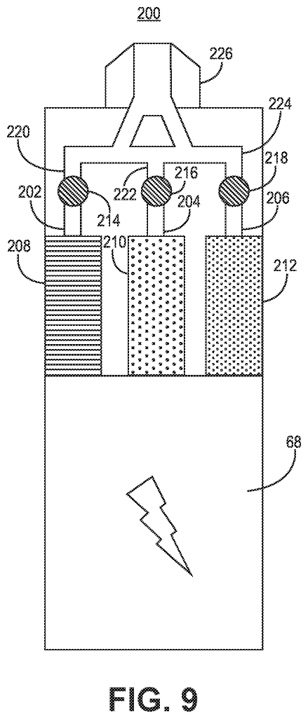

FIG. 9 shows another cross-sectional schematic of a multichannel vaporizer 200 with a different style of adjustable vapor manifold. In this case, rather than mixing through a central mixing valve, the vapor paths 202, 204, 206 leading to each cartridge 208, 210, 212 are individually regulated by valves 214, 216, 218. Vapor paths 220, 222, 224 exiting the valves 214, 216, 218 are combined prior to entering the mouthpiece 226. This configuration, while capable of being operated manually, may be best suited for computer control. Computer control in this configuration would more readily enable the ability to keep overall vapor content constant while varying the ratios of the individual formulations present in the vapor. Individual vapor path regulation can be accomplished by any methods known to those having skill in the art including manual or computer controlled, two-way valves, pinch valves, variable aperture iris valves, and the like.

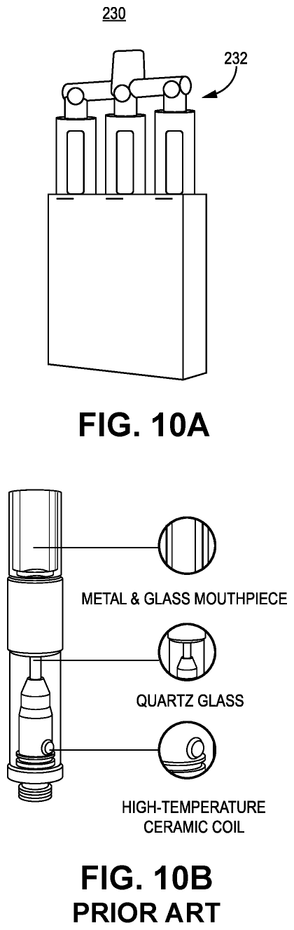

FIG. 10A depicts an embodiment of a multichannel vaporizer 230 depicting a tubular AVM 232, where the tubular portions are made up from materials such as, glass, metal, ceramic, or the like. In one example, tubular AVM 232 is transparent glass which allows the user to visualize vapor flow through the manifold. FIG. 10B illustrates a prior art representative of a vaporizer cartridge currently available on the market.

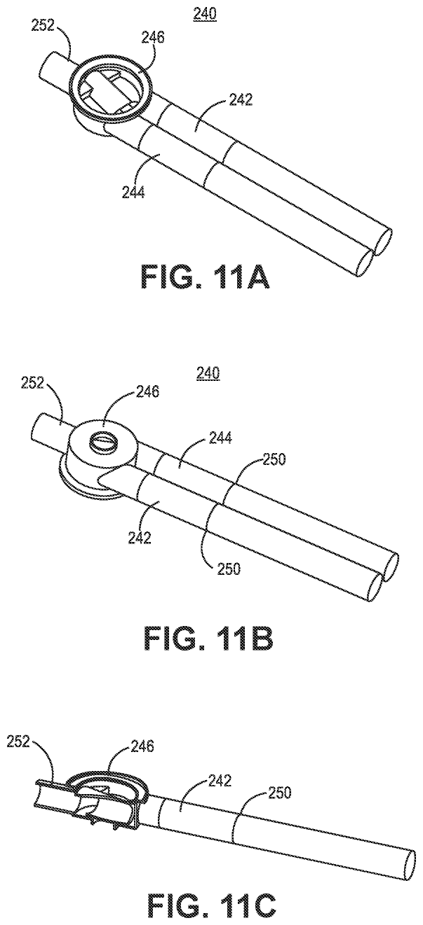

FIGS. 11A through 11C illustrates another embodiment of a multichannel vaporizer 240 having vapor cartridges 242, 244 and an adjustable vapor manifold 246 having cartridge engagement portions 250. FIG. 11A is a top view of the vaporizer 240. FIG. 11B is a bottom view of the vaporizer 240. FIG. 11C is a cross-sectional view of the vaporizer 240. Adjustable vapor manifold 246 includes an adjustable vapor regulator and at least one vapor path for communicating vapor from at least one cartridge to a mouthpiece 252. The vapor regulator can be adjusted to alter the ratio of vapor being delivered from each of the at least one cartridges 242, 244 thereby enabling a user to fine tune the composition of vapor. Cartridge engaging portions 250 may be sleeve-like to fit over a terminal end of a cartridge. Alternatively, cartridge engaging portions 250 may be adapted to nest inside a terminal end of a cartridge. Attachment devices may be employed at the cartridge engagement portions such as magnetic connectors, threading, and the like to make a more secure connection between vaporizer cartridges and the adjustable vapor manifold. Vaporizer cartridges specifically adapted for use with an adjustable vapor manifold are contemplated. Vapor paths are operatively coupled to the cartridge engagement portions. In other embodiments vapor flow regulators are incorporated into each vapor path. In some embodiments some or all of the vapor paths converge into adjustable flow regulators, such as multi-way valves. The flow regulators are adjustable via a control interface. The control interface may be manual or computer controlled. The control interface may provide continuous and/or discrete adjustments. For example, the control interface may have preset “stops” or settings that correspond to specific ratios of vapor from the plurality of cartridges. In some embodiments the adjustable vapor manifold may have, or connect to, supports for providing structural stability to a battery or batteries operatively coupled to the cartridge(s).

Vapor source cartridges to be used in the disclosed multichannel vaporizers may be any of the multitude of vaporizer cartridges known now to those having skill in the art or later invented. Generally, vaporizer cartridges have a main body portion that contains the vaporizable formulation, a heating element, a battery engaging portion, and a vapor output portion.

In some embodiments the ratio of vapor that reaches the user may be controlled by altering the power to each of the cartridges instead of via an adjustable vapor manifold.

Generally speaking the adjustable vapor manifold may be controlled mechanically or electrically. In the case where the AVM is controlled electronically, an input device such as a touch screen, provides signals to valve controllers (for example, via programmable logic controllers “PLC”) via a CPU executing coded commands. Pre-programmed ratios may be programmed into the CPU providing preset vapor formulations to users.

The Formulations: While the multichannel vaporizers can be used to vaporize any formulation adapted to function within a vaporizer cartridge, formulations containing cannabinoids and associated flavorants are particularly contemplated. Anticipated formulations include the major cannabinoids (THC and CBD), minor cannabinoids, terpenes, flavonoids, other flavoring agents, and other pharmacologically active ingredients. Cannabinoid extracts are known to those having skill in the art, such as, for example, super-/sub-critical CO2 extraction, hydrocarbon (pentane, butane, propane, etc), ethanol, and the like. Extracts containing other phytochemical moieties (“full spectrum” or “whole plant” extracts) may be used. More purified extracts, such as those created using distillation, may also be used. Combinations of extracts created using varied processes may also be combined. The term “vapor source” is not limited to vapor cartridges. As used herein, vapor source includes a variety of vapor cartridges and other vapor sources such as herbal vapes and “wax pen” style vapes.

In an example of a two-cartridge embodiment, the extracts used would be a THC extract (containing little to no CBD) and a CBD extract (containing little to no THC). These could be whole plant/full spectrum extracts or more highly purified distillation-based extracts that, in some embodiments, may be augmented with terpenes or other flavorants. In a specific embodiment a particular flavorant would be used for the THC and CBD extracts respectively such that the vapor from each would have a distinctive taste/flavor. For example, the THC extract might be predominantly flavored with myrcene while the CBD extract might be flavored with limonene. In such an embodiment a user would recognize the distinctive flavor profile associated with the THC and CBD extracts respectively and would therefore be able to recognize the taste of the vapor associated with different THC to CBD ratios. This would enable a method for providing a flavor-based signal to a user indicating the THC to CBD ratio of a product formulation.

In some embodiments, one of the formulations in one of the cartridges may serve to dilute the vapor being produced by cannabinoid and or “active ingredient” containing cartridges. Such a formulation may contain a vaporizer-safe excipient, i.e., PG, VG, MCT, or other excipients, or ingredients of marginal pharmacological activity known to those having skill in the art. This is useful in providing users with the ability to dial in the overall potency of the vapor delivered, by adjusting the amount of excipient being introduced into the vapor flow, without having to alter the ratio of actives delivered in the vapor.

In a particular embodiment the multichannel vaporizer comprises at least one battery operatively coupled to two vaporizer cartridges wherein a first vaporizer cartridge contains a vaporizable formulation of tetrahydrocannabinol and a second vaporizer cartridge contains a vaporizable formulation of cannabidiol, and wherein the vaporizable formulations further comprise a flavorant unique to the THC and CBD formulation respectively such as, for example, myrcene and linalool, but generally selected from the group consisting of terpenes and flavonoids. Generally the vaporizable formulations will contain between about 65 and 95% cannabinoid by weight, but particularly from between about 80-95% cannabinoid content by weight. In some embodiments the cannabinoid formulations are distillate. In some embodiments the cannabinoid formulations are CO2 or Ethanol extracts. The vaporizer cartridges are operatively coupled to an adjustable vapor manifold, such as, for example, by hollow vapor paths constructed from quartz glass tubes or inert metal tubes. The adjustable vapor manifold further comprises a 3 way mixing valve, either manually or computer controlled, that, depending on its functional position may let varying amounts of vapor from each of the cartridges through the manifold and out to a mouthpiece. The mixing valve may be continuous in operation or may be settable at discrete stops that correspond to distinct THC to CBD ratios, such as for example: 19:1, 6:1, 1:1, 1:6, and 1:19.

In embodiments the multichannel vaporizer may be provided in a kit comprising a multichannel vaporizer as described herein; a plurality of primary vaporizer cartridges each containing a vaporizable cannabinoid formulation, at least one but preferably a plurality of tertiary vaporizer cartridges containing flavorant formulations; a charging device, such as a USB cord; and instructions for using the AVM to titrate a dose that meets the user's needs. Kits may optionally include a carrying case.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. A vapor manifold for a multichannel vaporizer comprising:
a plurality of vapor sources; and
an adjustable valve connected to the plurality of vapor sources via a plurality of inlet vapor paths and connected to a mouthpiece via an outlet vapor path, with the valve being adjustable to modify a proportion of vapors from the plurality of vapor sources to be delivered to the mouthpiece.

2. The vapor manifold of claim 1 wherein the vapor source comprises a vaporizer cartridge.

3. The vapor manifold of claim 1 wherein the vapor source comprises a vapor chamber of a herbal vaporizer adapted for vaporization of plant material.

4. The vapor manifold of claim 3 wherein the plant material is cannabis.

5. The vapor manifold of claim 1 wherein the vapor source comprises the chamber of a botanical extract vaporizer adapted for vaporization of a botanical extract.

6. The vapor manifold of claim 5 wherein the botanical extract is cannabis extract.

7. The vapor manifold of claim 6 wherein the botanical extract is selected from the group consisting of shatter, budder, wax, live resin, rosin, isolate, crumble, hash, and honeycomb.

8. A vaporizer device comprising:
a lower unit including a battery power source and carrying a plurality of vapor sources;
an upper unit including a mouthpiece for issuing a combination of vapors from the plurality of vapor sources; and
an adjustable vapor manifold including a plurality of vapor paths from the plurality of vapors sources to at least one valve for mixing said vapors to produce the combination of vapors to be issued via the mouthpiece.

9. The vapor manifold of claim 8 wherein the vapor source comprises a vaporizer cartridge.

10. The vapor manifold of claim 8 wherein the vapor source comprises a vapor chamber of a herbal vaporizer adapted for vaporization of plant material.

11. The vapor manifold of claim 10 wherein the plant material is cannabis.

12. The vapor manifold of claim 8 wherein the vapor source comprises the chamber of a botanical extract vaporizer adapted for vaporization of a botanical extract.

13. The vapor manifold of claim 12 wherein the botanical extract is cannabis extract.

14. The vapor manifold of claim 13 wherein the botanical extract is selected from the group consisting of shatter, budder, wax, live resin, rosin, isolate, crumble, hash, and honeycomb.

15. A vaporizer device comprising:
a plurality of vapor sources;
a mouthpiece for issuing a combination of vapors from the plurality of vapor sources; and
an adjustable vapor manifold defining a plurality of vapor paths from the plurality of vapors sources to at least one valve for mixing said vapors to produce the combination of vapors to be issued via the mouthpiece.

16. The vaporizer device of claim 15 wherein the at least one valve is either manually or electrically operated.

17. The vaporizer device of claim 15 wherein the adjustable vapor manifold is controlled via a user interface.

18. The vaporizer device of claim 17 wherein the user interface provides predetermined stops defining different proportions of vapors from the vapor sources.
